(19) United States
(12) Patent Application Publication
(10) Pub. No.: US 2026/0306087 A1
(43) Pub. Date: Oct. 8, 2026

(54) Abdominal Wall Retractor

(71) Applicant: Clyde Jacob, Duluth, GA (US)

(72) Inventor: Clyde Jacob, Duluth, GA (US)

(21) Appl. No.: 19/481,489

(22) PCT Filed: May 3, 2024
(86) PCT No.: PCT/US2024/027690

§ 371 Date: Nov. 4, 2025

Related U.S. Application Data
(60) Provisional application No. 63/500,323, filed on May 5, 2023.

(51) Int. Cl. A61B 17/02; A61B 90/57

(52) U.S. Cl. CPC A61B 17/02; A61B 90/57; A61B 2090/571

(57) ABSTRACT

A retractor system comprising a retractor blade, a handle secured to the retractor blade, and a support assembly having a transverse wing member rigidly coupled to the handle, at least one bracket configured to engage a respective portion of the transverse wing member, and at least one extension arm, wherein each extension arm is coupled to a respective bracket and at least one clamp configured to be rigidly secured to a surgical bed and configured to engage a respective extension arm.

### CROSS-REFERENCE TO RELATED APPLICATION

The application claims priority to U.S. Provisional Patent Application No. 63/500,323, filed May 5, 2023, the entirety of which is incorporated by reference herein.

### FIELD

The application is generally related to surgical devices and procedures and, more particularly, to retractors and methods of use thereof that facilitate access to a patient's abdominal cavity.

### BACKGROUND

Various surgical procedures require that the performing surgeon gain access to the abdominal cavity of a patient. It is well known to use devices, such as retractors, to urge the abdominal wall outwardly from the primary incision site so that the surgeon may have unfettered access to the abdominal cavity during the procedure. The most basic types of retractors provide a structure to engage the abdominal wall adjacent the primary incision and exert force outwardly by way of grasping a handle. Although effective, such devices require constant outward force be exerted on the retractor, which can lead to fatigue by the user or require that multiple personnel take turns maintaining access to the abdominal cavity.

From the foregoing, one recognizes the need for retractors that may be utilized during extended procedures that require little to no physical exertion to maintain access to the abdominal cavity.

### SUMMARY

Embodiments of the disclosed abdominal wall retractor are configured to allow a user to maintain access to a patient's abdominal cavity without the need for an individual to apply constant force to the retractor. In some aspects, the disclosed abdominal wall retractor can include a retractor blade, a handle secured thereto, and a support assembly allowing the position of the retractor within the patient to be maintained by securing the retractor to a fixed structure, such as a portion of a surgical table. The support assembly may include a pair of extension arms extending inwardly toward the patient, the extension arms being secured to the surgical table at their distal ends by adjustable clamps. At proximal ends of the extension arms, brackets secure the arms to a wing member that is coupled to the handle of the retractor. The brackets and clamps allow for rapid and secure adjustment of the retractors with respect to the patient's abdominal cavity. Because the retractor may be secured in the desired position, constant force need not be applied by the user, therefore reducing the potential for fatigue.

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended drawings wherein:

FIG. 1 is an isometric view of an embodiment of an abdominal wall retractor system in accordance with an embodiment of the present invention.

FIG. 2 is an isometric representation of an extension arm of the support assembly of the abdominal wall retractor system shown in FIG. 1.

FIG. 3 is an isometric view of a clamp of the support assembly of the abdominal wall retractor system shown in FIG. 1.

FIG. 4 is a perspective view of an embodiment of an abdominal wall retractor system in accordance with an embodiment of the present invention.

FIG. 5 is a partial perspective view of the retractor system, with the retractor in a first position relative to the support assembly.

FIG. 6 is a partial perspective view of the retractor system of FIG. 5, with the retractor in a pivoted position for movement relative to the support assembly.

FIG. 7 is a partial perspective view of the retractor system of FIG. 5, with the retractor moved to a second position relative to the support assembly.

FIG. 8 is a partial perspective view of the retractor system of FIG. 5, with the retractor in the second position, pivoted to inhibit movement relative to the support assembly.

FIG. 9 is a perspective view of an exemplary bracket as disclosed herein.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a,” “an,” and “the” include plural referents unless the context clearly dictates otherwise. For example, use of the term “a member” can represent disclosure of embodiments that include a single such member, as well as disclosure of embodiments that include a plurality of such members, and so forth.

All technical and scientific terms used herein have the same meaning as commonly understood to one of ordinary skill in the art to which this invention belongs unless clearly indicated otherwise.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of.” For example, “at least one of A, B and C” explicitly includes only A, only B, only C, and combinations of each.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedents “about,” “substantially,” or “generally,” it is contemplated that values within up to 15%, up to 10%, up to 5%, or up to 1% (above or below) of the particularly stated value can be included within the scope of those aspects. In other aspects, when angular values are approximated by use of the antecedents “about,” “substantially,” or “generally,” it is contemplated that angular values within up to 15 degrees, up to 10 degrees, up to 5 degrees, or up to one degree (above or below) of the particularly stated angular value can be included within the scope of those aspects.

The word “or” as used herein means any one member of a particular list and, unless context dictates otherwise, can also represent disclosure of embodiments that include any combination of members of that list.

In the following description and claims, wherever the word “comprise” or “include” is used, it is understood that the words “comprise” and “include” can optionally be replaced with the words “consists essentially of” or “consists of” to form another embodiment.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

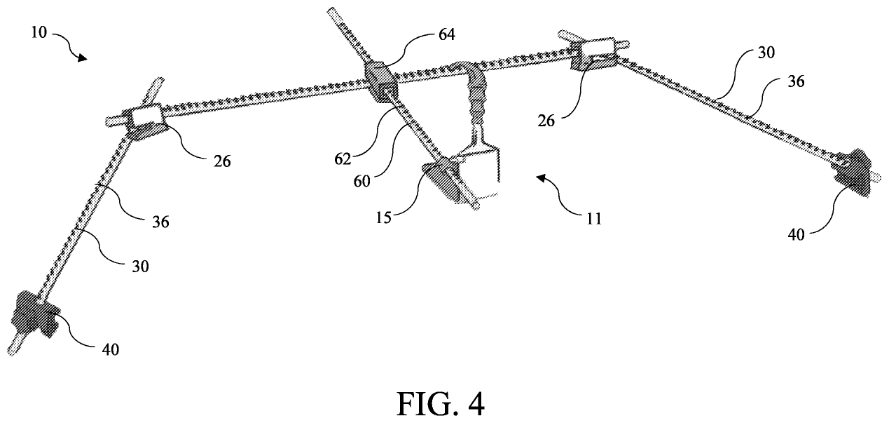

Referring now to the Figures, an abdominal wall retractor assembly 10 in accordance with an embodiment of the present disclosure includes a retractor 11 comprising a retractor blade 12 and a handle 14 extending upwardly from a first end of the retractor blade. The retractor assembly 10 can further comprise a support assembly 20 for securing the retractor assembly to a stationary object once access to a patient's abdominal cavity is obtained. As shown, the retractor blade 12 is substantially planar and includes a rake 16 disposed along the edge of the first end to which the handle 14 is secured. The rake 16 helps maintain the position of the retractor by engaging the abdominal wall along the perimeter of the incision. In other aspects, and as illustrated in FIG. 4, the rake 16 can be omitted. In some aspects, the handle 14 can include a contoured grip to facilitate grasping by the user while applying outward force to the abdominal wall of the patient.

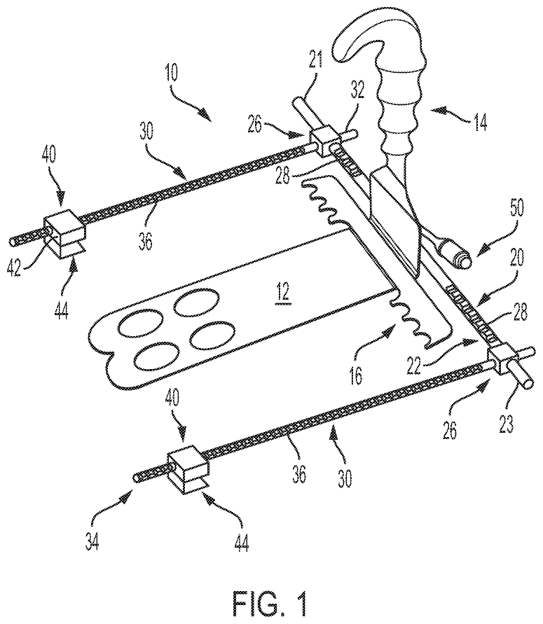

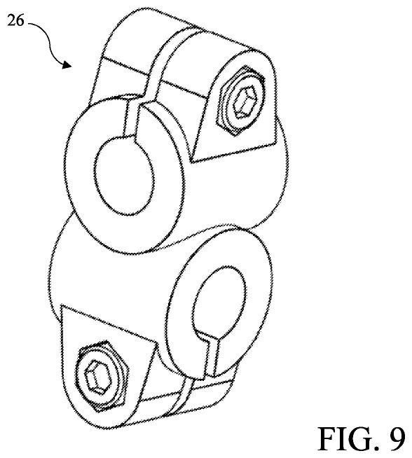

As shown in FIG. 1, the support assembly 20 can include an elongate transverse wing member 22 that is coupled to the retractor 11. The wing member 22 includes a pair of opposed end portions 21 and 23 that each receive a bracket 26 thereon. Each bracket 26 can be configured to be secured in a fixed position along one of the corresponding end portions 21 and 23 of the wing member 22. For example, the transverse wing member 22 can comprise a ratchet 28 (e.g., a plurality of notches/grooves), and the brackets 26 can each comprise a pawl configured to engage the ratchet 28 to secure the bracket in a fixed position along the wing member 22. That is, each bracket 26 can selectively engage at least one of a plurality of grooves 28 disposed along the wing member 22 to temporarily lock each bracket 26 in the desired position. As such, each bracket 26 may be moved either closer to or farther from the handle 14 independently of the other bracket 26. In some aspects, each bracket 26 can have a release actuator (e.g., a button or lever) that disengages the pawl of the bracket from the ratchet 28 in order to permit movement of the bracket 26 along the wing member 22. The release actuator can be spring-biased toward an engagement configuration so that the pawl engages the ratchet 28 when a user is not actuating the release actuator. In additional aspects, the bracket 26 can be adjustably secured to the wing member 22 in a plurality of different positions using other fastening mechanisms, such as screw clamps or ball detents. For example, FIG. 9 illustrates an exemplary clamp 26 defining a pair of through-bores that are angularly offset (e.g., by 90 degrees) and laterally offset. As illustrated, a set screw can be tightened down to reduce the operative diameter of the respective bore to tighten the clamp 26 against a member extending through the bore (e.g., the elongate transverse wing member 22). In other aspects, a screw coupled to a handle (such as that of a C-clamp) or a clamp can be used to secure the member to the clamp 26. Although the through-bores shown in FIG. 9 are cylindrical, it is contemplated that other geometries (e.g., square, rectangular, or elongate slot-shaped bores) can be used for receiving the member therethrough.

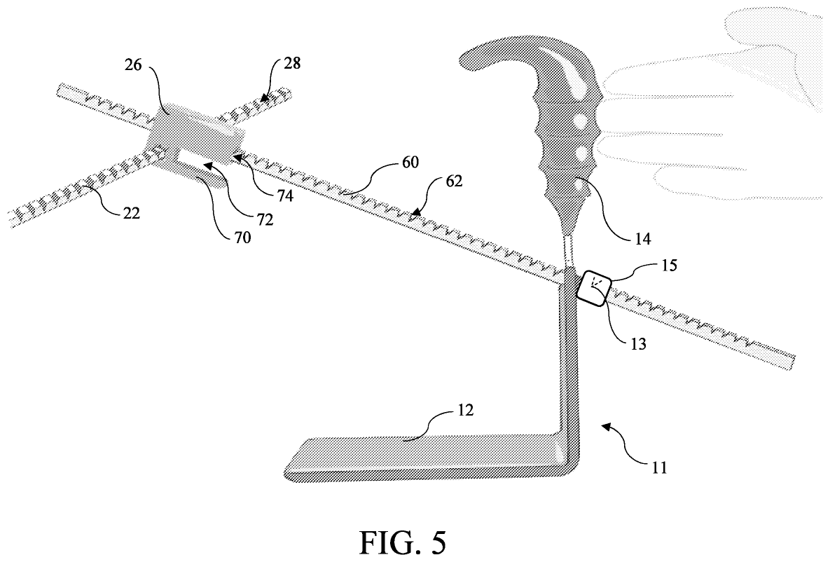

Referring to FIG. 5, in some optional aspects, each bracket 26 can comprise a first portion and a separable second portion. Each of the first and second separable portions 26 can comprise a leg 70 and a slot 72. The leg 70 of the first portion can be received with in the slot 72 of the second portion so that the first and second portions are coupled together. Each of the first and second portions can each define a bore 74 therethrough for receiving and engaging a respective member. Accordingly, the first and second portions can cooperate to define a bracket 26, with the respective bores being angularly and laterally offset for coupling the respective members. In other aspects, the bracket can comprise a unitary body defining two bores 74 therethrough, wherein said bores 74 are laterally offset and angularly offset (e.g., by about 90 degrees).

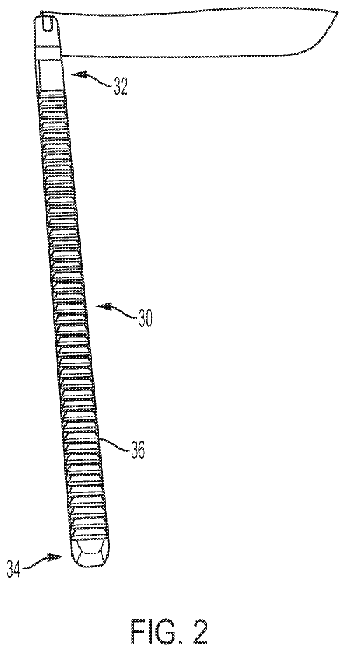

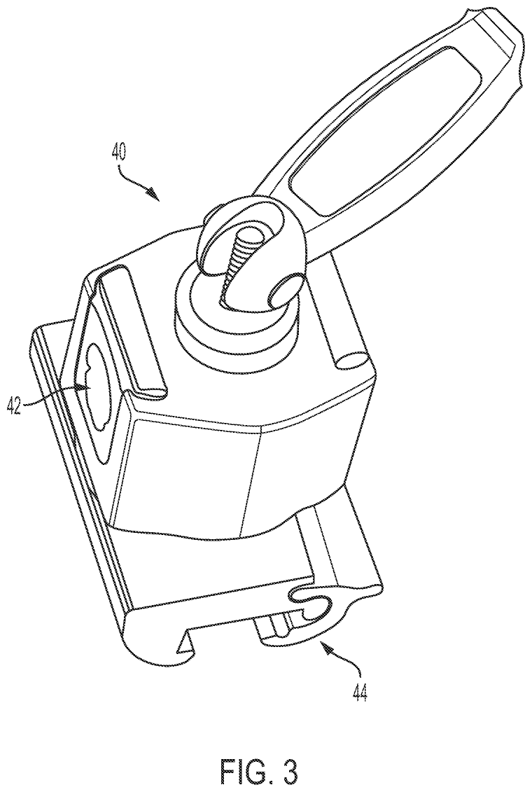

Referring also to FIG. 2, the support assembly 20 also includes a pair of extension arms 30. Each extension arm 30 includes a proximal end 32 that is secured to a corresponding arm 21 and 23 of the wing member 22 by a corresponding bracket 26, and a distal end 34 that is slidably received in by a corresponding clamp 40. A protrusion 42 of each clamp is configured to selectively engage a ratchet 36 formed along each extension arm 30. For example, each clamp 40 can comprise a pawl that is configured to slidably receive the distal end 34 of a corresponding extension arm 30. Each clamp 40 can include a grip portion 44 that is configured to be releasably secured to a fixed object, such as a portion of a surgical bed. In some aspects, each clamp 40 can be configured to swivel over an angular range of at least 90° to permit for adjusting the retractor blade 12.

In some aspects, and as illustrated in FIG. 1, the retractor 11 can be rigidly coupled to the wing member 22, and the position of the retractor can be adjusted along a transverse axis by moving the wing member 22 relative to the brackets 26, and along longitudinal axis by moving the wing member 22 relative to the extension arms 30 (e.g., by adjusting the position of the extension arms 30 in the clamps 40).

In other aspects, the support assembly 20 can further comprise a support arm 60 that couples to and extends from the transverse wing member 22. The support arm can comprise a first ratchet 62. The retractor 11 can comprise a pawl 13 (FIG. 5) that is configured to engage the ratchet 62 of the support arm. For example, the retractor 11 can comprise a sleeve 15 (shown as transparent in FIG. 5 to show the pawl 13) that surrounds the support arm 60, and the pawl 13 can be positioned within the sleeve 15.

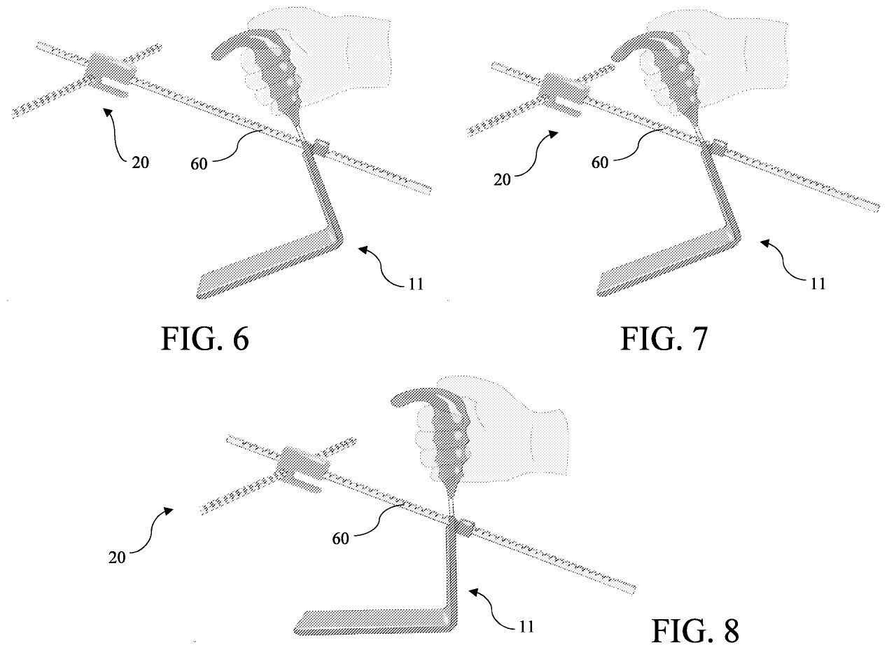

Referring to FIGS. 5-8, to adjust the position of the retractor 11, a medical professional can grip the handle 14 to tilt the retractor forwardly to disengage the pawl 13 from the first ratchet 62. The retractor 11 can then slide along the support arm 60 to a desired position. The medical professional can tilt the retractor 11 rearwardly to engage the pawl 13 with the first ratchet 62 to thereby secure the retractor in place.

The support assembly 20 can further comprise a wing member bracket 64 that couples the support arm 60 to the transverse wing member 22. For example, as described herein, the transverse wing member 22 can comprise a ratchet 28. The wing member bracket 64 can be configured to engage the ratchet 28 of the transverse wing member 22 to fix a longitudinal position of the wing member bracket 64 therealong. In other aspects, the wing member bracket 64 can engage the transverse wing member 22 and the support arm 60 as shown and described with reference to FIG. 9, wherein the member/arm extends through a respective bore, and the bore is tightened down against the member/arm.

Accordingly, in some aspects, the clamps 40 can be secured to the hospital bed. The extension arms 30 can be coupled to the clamps 40. The transverse wing member 22 can be coupled to extension arms 30. A position of the wing member bracket 64 can be selected along the transverse wing member 22, thereby selecting the position of the support arm 60. In some aspects, the position of the support arm 60 need not be adjusted during surgery. Accordingly, in some aspects, during surgery, the medical professional need only modify the position of the retractor relative to the support arm 60.

In some aspects, the blade 12 of the retractor 11 can extend along a first axis, and the support arm 60 can extend along a second axis that is oriented at an acute angle to the first axis. Optionally, the second axis along which the support arm 60 extends can be oriented at an acute angle relative to a horizontal axis so that forward movement of the retractor effects an upward movement of the blade, thereby lifting the incision.

As shown in FIG. 1, the retractor assembly 10 includes a fiber optics port 50 to facilitate use of fiber optic monitoring systems in the vicinity of the abdominal cavity. Preferably, all components of the retractor assembly 10 are constructed of surgical stainless steel to support cleansing by autoclaving.

It is contemplated that the relative dimensions shown in the figures can differ based on the configuration of the environment in which the retractor assembly 10 is used. For example, the extension arms 30 can be longer than the support arm 60.

### EXEMPLARY ASPECTS

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A retractor system comprising:
a retractor comprising:
   a retractor blade; and
   a handle secured to the retractor blade; and
a support assembly comprising:
   a transverse wing member;
   at least one extension arm coupled to the transverse wing member; and
   at least one clamp configured to be rigidly secured to a surgical bed,
wherein each clamp of the at least one clamp is configured to engage a respective extension arm of the at least one extension arm.
   wherein the retractor is coupled to the support assembly so that the support assembly maintains the retractor in a fixed position relative to the surgical bed.

Aspect 2: The retractor system of aspect 1, wherein the retractor is releasably coupled to the support assembly to permit adjustment of the fixed position.

Aspect 3: The retractor system of aspect 2, wherein the support assembly further comprises a support arm that couples to and extends from the transverse wing member, wherein the support arm comprises a first ratchet, wherein the retractor comprises a pawl that is configured to engage the first ratchet to releasably couple to the support assembly in the fixed position.

Aspect 4: The retractor system of aspect 3, wherein the transverse wing member comprises a second ratchet.

Aspect 5: The retractor system of aspect 4, further comprising a bracket that is configured to engage the transverse wing member to fix a longitudinal position therealong, wherein the bracket couples the support arm to the transverse wing member.

Aspect 6: The retractor system of any one of the preceding aspects, wherein the retractor is rigidly coupled to the transverse wing member.

Aspect 7: The retractor system of any one of the preceding aspects, further comprising at least one bracket, wherein each bracket of the at least one bracket is configured to couple a respective extension arm of the at least one extension arm to the transverse wing member.

Aspect 8: The retractor system of aspect 7, wherein each extension arm comprises a respective ratchet, wherein each bracket of the at least one bracket is configured to engage the respective ratchet of the respective extension arm.

Aspect 9: The retractor system of any one of the preceding aspects, wherein the at least one extension arm comprises a ratchet, wherein each clamp of the at least one clamp of the support assembly comprises a clamp protrusion that engages the ratchet of the respective extension arm of the at least one extension arm.

Aspect 10: The retractor system of aspect 9, wherein the ratchet of each extension arm of the at least one extension arm comprises a plurality of grooves, and wherein the clamp ratchet of each clamp of the at least one clamp of the support assembly is configured to sequentially engage respective grooves of the plurality of grooves of the respective extension arm of the at least one extension arm.

Aspect 11: The retractor system of any one of the preceding aspects, wherein each clamp of the at least one clamp comprises a swivel that permits movement within an angular range of at least 90 degrees.

Aspect 12: The retractor system of any one of the preceding aspects, wherein the support assembly permits rotation of the transverse wing member by at least 180 degrees about a horizontal axis to permit adjustment of an angular orientation of the retractor blade.

Aspect 13: The retractor system any one of the preceding aspects, wherein the transverse wing member of the support assembly has opposed first and second ends, and wherein the at least one ratchet comprises a first ratchet configured to engage the first end of the transverse wing member and a second ratchet configured to engage the second end of the transverse wing member.

Aspect 14: The retractor system of aspect 13, wherein the at least one extension arm comprises a first extension arm that is coupled to the first ratchet and a second extension arm that is coupled to the second ratchet, and wherein the at least one clamp comprises a first clamp that is configured to engage the first extension arm and a second clamp that is configured to engage the second extension arm.

Aspect 15: The retractor system of any one of the preceding aspects, further comprising a fiber optics port coupled to the handle.

Aspect 16: The retractor system of any one of the preceding aspects, further comprising at least one rake coupled to the retractor blade.

Aspect 17: The retractor system of any one of the preceding aspects, wherein the transverse wing member comprises surgical steel.

Aspect 18: A method of using the system of any one of the preceding aspects, wherein the method comprises using the handle to rotate the retractor blade, wherein following release of the handle, the at least one ratchet of the support assembly maintains an angular orientation of the transverse wing member to thereby maintain a position of the retractor blade.

Aspect 19: The method of aspect 18, further comprising using the at least one clamp to provide leverage to the support assembly during rotation of the retractor blade.

Aspect 20: The method of aspect 18 or aspect 19, wherein the at least one clamp is positioned laterally of a surgical site.

Aspect 21: The method of any one of aspects 18-20, further comprising positioning a plastic cover over at least a portion of the support assembly to protect a sterile field.

Aspect 22: A retractor apparatus comprising:
a retractor blade;
a handle secured to the retractor blade; and
a support assembly having:
   a transverse wing member rigidly coupled to the handle, wherein the transverse wing member comprises a ratchet; and
   at least one bracket, wherein each bracket of the at least one bracket is configured to engage a respective portion of the transverse wing member.

Aspect 23: The retractor apparatus of aspect 22, further comprising a fiber optics port coupled to the handle.

Aspect 24: The retractor apparatus of aspect 22 or aspect 23, further comprising at least one rake coupled to the retractor blade.

Aspect 25: The retractor apparatus of any one of aspects 22-24, wherein the transverse wing member comprises surgical steel.

Although the foregoing invention has been described in some detail by way of illustration and example for purposes of clarity of understanding, certain changes and modifications may be practiced within the scope of the appended claims.

1. A retractor system comprising:
a retractor comprising:
a retractor blade; and
a handle secured to the retractor blade; and
a support assembly comprising:
a transverse wing member;
at least one extension arm coupled to the transverse wing member; and
at least one clamp configured to be rigidly secured to a surgical bed, wherein each clamp of the at least one clamp is configured to engage a respective extension arm of the at least one extension arm,
wherein the retractor is coupled to the support assembly so that the support assembly maintains the retractor in a fixed position relative to the surgical bed.

2. The retractor system of claim 1, wherein the retractor is releasably coupled to the support assembly to permit adjustment of the fixed position.

3. The retractor system of claim 2, wherein the support assembly further comprises a support arm that couples to and extends from the transverse wing member, wherein the support arm comprises a first ratchet, wherein the retractor comprises a pawl that is configured to engage the first ratchet to releasably couple to the support assembly in the fixed position.

4. The retractor system of claim 3, wherein the transverse wing member comprises a second ratchet.

5. The retractor system of claim 4, further comprising a bracket that is configured to engage the transverse wing member to fix a longitudinal position therealong, wherein the bracket couples the support arm to the transverse wing member.

6. The retractor system of claim 1, wherein the retractor is rigidly coupled to the transverse wing member.

7. The retractor system of claim 1, further comprising at least one bracket, wherein each bracket of the at least one bracket is configured to couple a respective extension arm of the at least one extension arm to the transverse wing member.

8. The retractor system of claim 7, wherein each extension arm comprises a respective ratchet, wherein each bracket of the at least one bracket is configured to engage the respective ratchet of the respective extension arm.

9. The retractor system of claim 1, wherein the at least one extension arm comprises a ratchet, wherein each clamp of the at least one clamp of the support assembly comprises a clamp protrusion that engages the ratchet of the respective extension arm of the at least one extension arm.

10. The retractor system of claim 9, wherein the ratchet of each extension arm of the at least one extension arm comprises a plurality of grooves, and wherein the clamp ratchet of each clamp of the at least one clamp of the support assembly is configured to sequentially engage respective grooves of the plurality of grooves of the respective extension arm of the at least one extension arm.

11. The retractor system of claim 1, wherein each clamp of the at least one clamp comprises a swivel that permits movement within an angular range of at least 90 degrees.

12. The retractor system of claim 1, wherein the support assembly permits rotation of the transverse wing member by at least 180 degrees about a horizontal axis to permit adjustment of an angular orientation of the retractor blade.

13. The retractor system claim 1, wherein the transverse wing member of the support assembly has opposed first and second ends, and wherein the at least one ratchet comprises a first ratchet configured to engage the first end of the transverse wing member and a second ratchet configured to engage the second end of the transverse wing member.

14. The retractor system of claim 13, wherein the at least one extension arm comprises a first extension arm that is coupled to the first ratchet and a second extension arm that is coupled to the second ratchet, and wherein the at least one clamp comprises a first clamp that is configured to engage the first extension arm and a second clamp that is configured to engage the second extension arm,

15. The retractor system of claim 1, further comprising a fiber optics port coupled to the handle.

16. The retractor system of claim 1, further comprising at least one rake coupled to the retractor blade.

17. The retractor system of claim 1, wherein the transverse wing member comprises surgical steel.

18. A method of using the system of claim 1, wherein the method comprises using the handle to rotate the retractor blade, wherein following release of the handle, the at least one ratchet of the support assembly maintains an angular orientation of the transverse wing member to thereby maintain a position of the retractor blade.

19. (canceled)

20. (canceled)

21. (canceled)

22. A retractor apparatus comprising:
a retractor blade;
a handle secured to the retractor blade; and
a support assembly having:
a transverse wing member rigidly coupled to the handle, wherein the transverse wing member comprises a ratchet; and
at least one bracket, wherein each bracket of the at least one bracket is configured to engage a respective portion of the transverse wing member.

23. The retractor apparatus of claim 22, further comprising a fiber optics port coupled to the handle.

24. (canceled)

25. (canceled)
